(19) United States
(12) Patent Application Publication
(10) Pub. No.: US 2022/0096487 A1
(43) Pub. Date: Mar. 31, 2022

(54) METHODS FOR TREATING HIV WITH DOLUTEGRAVIR AND LAMIVUDINE

(71) Applicant: VIIV HEALTHCARE COMPANY, Wilmington, DE (US)

(72) Inventor: Martin John GARTLAND, Research Triangle Park, NC (US)

(73) Assignee: VIIV HEALTHCARE COMPANY, Wilmington, DE (US)

(21) Appl. No.: 17/426,146

(22) PCT Filed: Feb. 3, 2020
(86) PCT No.: PCT/US2020/016321

§ 371 Date: Jul. 28, 2021

Related U.S. Application Data
(60) Provisional application No. 62/801,243, filed on Feb. 5, 2019.

(51) Int. Cl. A61K 31/5365; A61K 31/506; A61K 31/155; A61K 33/24; A61K 33/26; A61P 31/18

(52) U.S. Cl. CPC A61K 31/5365; A61K 31/506; A61P 31/18; A61K 33/24; A61K 33/26; A61K 31/155

(57) ABSTRACT

Invented are methods for treating HIV in a human in need thereof which comprises the administration of a therapeutically effective amount of a combination of dolutegravir or a pharmaceutically acceptable salt thereof and lamivudine or a pharmaceutically acceptable salt thereof, to such human.

### FIELD OF THE INVENTION

This invention relates to a method of treating HIV in a human by the in vivo administration of dolutegravir or a pharmaceutically acceptable salt thereof, in combination with lamivudine or a pharmaceutically acceptable salt thereof.

### BACKGROUND OF THE INVENTION

The contemporary standard of care for first-line treatment of HIV-1 infection in adults naive to antiretroviral therapy (ART) is a regimen of >3 antiretroviral agents that includes two nucleoside reverse transcriptase inhibitors (NRTIs) and one other drug from either the boosted protease inhibitor (PI), integrase strand transfer inhibitor (INSTI), or non-nucleoside reverse transcriptase inhibitor (NNRTI) classes. However, concerns exist regarding the need for lifelong therapy with drugs that have diverse safety and tolerability profiles. Thus, 2-drug regimens (2DRs) capable of inducing and/or maintaining virologic suppression while decreasing lifetime cumulative drug exposure and potential long-term toxicities would represent an alternative treatment option for people living with HIV-1 infection.

Trials evaluating early (2000-2014) 2DRs yielded inconclusive results, perhaps partially because of small sample sizes, short treatment durations, and limitations of available treatments. The AIDS Clinical Trials Group Study A5142 team found that the virologic efficacy of a 2DR comprising efavirenz plus ritonavir-boosted lopinavir (n=250) was similar to that of efavirenz plus 2 NRTIs (n=250) through 96 weeks of treatment in ART-naive participants. However, this 2DR was associated with increased incidence of drug resistance. Likewise, the PROGRESS study demonstrated that a 2DR of ritonavir-boosted lopinavir plus raltegravir (n=101) exerts a similar antiviral effect in ART-naive participants through Week 96 compared with the 3-drug regimen (3DR) of ritonavir-boosted lopinavir plus tenofovir disoproxil fumarate/emtricitabine (n=105), but this study mostly enrolled participants with viral loads <100,000 copies/mL and CD4+ cell counts ≥200 cells/mm. The GARDEL study demonstrated the non-inferior virologic efficacy of open-label ritonavir-boosted lopinavir plus lamivudine (n=214) compared with ritonavir-boosted lopinavir plus 2 NRTIs (n=202) in ART-naive participants after 48 weeks of treatment. However, these 2DRs include ritonavir-boosted PIs, which are associated with a variety of metabolic syndromes and cardiovascular-related disease and may negate any anticipated benefit in terms of decreased drug exposure and cumulative toxicity. These studies illustrate both the potential of 2DRs as an option for ART and the importance of selecting drugs with appropriate and complementary virologic and clinical properties.

The INSTI dolutegravir has emerged as a strong candidate for a core agent in 2DRs. The phase III clinical development of dolutegravir has provided extensive evidence, with up to 144 weeks of follow-up data supporting the virologic efficacy, favourable tolerability profile, and high barrier to resistance as a core agent in first-line 3DRs in ART-naive individuals. In the SWORD-1 and SWORD-2 studies, dolutegravir plus rilpivirine exhibited non-inferior virologic efficacy for maintaining virologic suppression and acceptable tolerability compared with the continuation of current ART through Week 48. Week 100 analysis showed that the high rate of virologic suppression was maintained, with a low discontinuation rate.

Lamivudine is an NRTI included in several standard-care regimens for first-line treatment of HIV-1 infection. Trials evaluating lamivudine have demonstrated well-established efficacy, safety, and tolerability profiles. One challenge associated with lamivudine is its rapid development of drug resistance, resulting from selection of the M184V reverse transcriptase mutation when used as monotherapy. However, results from in vitro studies have suggested that lamivudine, when combined with other agents, retains a portion of its antiviral potency after selection of M184V variants. The 2DR of dolutegravir plus lamivudine was evaluated in the 48-week pilot study PADDLE for the treatment of ART-naive participants. By Week 8, all 20 participants, including four with baseline HIV-1 RNA >100,000 copies/mL, had achieved a viral load of <50 copies/mL; 90% (n=18) maintained virologic suppression at Week 48, and all 18 participants maintained virologic suppression at Week 96. Additionally, the phase II, single-arm ACTG A5353 study in 120 treatment-naive participants with HIV-1 RNA <500,000 copies/mL treated with dolutegravir plus lamivudine demonstrated that 90% (n=108) achieved HIV-1 RNA <50 copies/mL at Week 24. Thus, dolutegravir plus lamivudine may constitute a viable combination as a novel 2DR for use in initial suppression of HIV-1 infection in ART-naive patients. See Calm et al., “Dolutegravir plus lamivudine versus dolutegravir plus tenofovir disoproxil fumarate and emtricitabine in antiretroviral-naive adults with HIV-1 infection (GEMINI-1 and GEMINI-2): week 48 results from two multicentre, double-blind, randomised, non-inferiority, phase 3 trials.”, Volume 393, Issue 10167, January 12, 2019: 143-155.

### SUMMARY OF THE INVENTION

This invention comprises a method of treating HIV in a human in need thereof which comprises the in vivo administration of a therapeutically effective amount of a combination of: dolutegravir or a pharmaceutically acceptable salt thereof, and lamivudine or a pharmaceutically acceptable salt thereof.

According to another embodiment, the invention comprises dolutegravir or a pharmaceutically acceptable salt thereof, and lamivudine or a pharmaceutically acceptable salt thereof for use in therapy.

According to another embodiment, the invention comprises dolutegravir or a pharmaceutically acceptable salt thereof, and lamivudine or a pharmaceutically acceptable salt thereof for use in the treatment of HIV.

According to another embodiment, the invention comprises the use of dolutegravir or a pharmaceutically acceptable salt thereof, and lamivudine or a pharmaceutically acceptable salt thereof in the preparation of a medicament for the treatment of HIV.

This invention consists essentially of a method of treating HIV in a human in need thereof which comprises the in vivo administration of a therapeutically effective amount of a combination of: dolutegravir or a pharmaceutically acceptable salt thereof, and lamivudine or a pharmaceutically acceptable salt thereof.

According to another embodiment, the invention consists essentially of dolutegravir or a pharmaceutically acceptable salt thereof, and lamivudine or a pharmaceutically acceptable salt thereof for use in therapy.

According to another embodiment, the invention consists essentially of dolutegravir or a pharmaceutically acceptable salt thereof, and lamivudine or a pharmaceutically acceptable salt thereof for use in the treatment of HIV.

According to another embodiment, the invention consists essentially of the use of dolutegravir or a pharmaceutically acceptable salt thereof, and lamivudine or a pharmaceutically acceptable salt thereof in the preparation of a medicament for the treatment of HIV.

### DETAILED DESCRIPTION OF THE INVENTION

The combination, a two-drug combination of dolutegravir (integrase strand transfer inhibitor [INSTI]) and lamivudine (nucleoside analogue reverse transcriptase inhibitor [NRTI]) is indicated for the treatment of human immunodeficiency virus type 1 (HIV-1) infection in adults with no known substitutions associated with resistance to the individual components of the combination.

1 Indications and Usage

The combination is indicated for the treatment of human immunodeficiency virus type 1 (HIV-1) infection in adults with no known substitutions associated with resistance to the individual components of the combination.

2 Dosage and Administration

2.1 Pregnancy Testing Before Initiation of the Combination

Perform pregnancy testing before initiation of the combination in adolescents and adults of childbearing potential [see Warnings and Precautions (5.6), Use in Specific Populations (8.1, 8.3)].

2.2 Recommended Dosage

The combination is a fixed-dose combination product containing 50 mg of dolutegravir and 300 mg of lamivudine. The recommended dosage regimen of the combination in adults is one tablet taken orally once daily with or without food.

2.3 Recommended Dosage with Certain Concomitant Medications

The dolutegravir dose (50 mg) in the combination is insufficient when coadministered with medications listed in Table 1 that may decrease dolutegravir concentrations; the following dolutegravir dosage regimen is recommended.

TABLE 1
Dosing Recommendations for the combination
with Coadministered Medications

| Coadministered Drug | Dosing Recommendation |
|---|---|
| Carbamazepine, rifampin | The recommended dolutegravir dosage |
| | regimen is 50 mg twice daily. An additional |
| | dolutegravir 50-mg tablet, separated by 12 |
| | hours from the combination, should be taken. |

2.4 Not Recommended Due to Lack of Dosage Adjustment

Because the combination is a fixed-dose tablet and cannot be dose adjusted, the combination is not recommended in patients with creatinine clearance less than 50 mL per minute [see Use in Specific Populations (8.6)].

3 Dosage Forms and Strengths

The combination tablets are oval, biconvex, white, film-coated tablets, debossed with “SV 137” on one face. Each tablet contains dolutegravir sodium equivalent to 50 mg of dolutegravir and 300 mg of lamivudine [see Description (11)].

4 Contraindications

The combination is contraindicated in patients with prior hypersensitivity reaction to dolutegravir [see Warnings and Precautions (5.2)] or lamivudine, or receiving dofetilide, due to the potential for increased dofetilide plasma concentrations and the risk for serious and/or life-threatening events with concomitant use of dolutegravir [see Drug Interactions (7)].

5 Warnings and Precautions

5.1 Posttreatment Exacerbations of Hepatitis in Patients with Hepatitis B Co-Infection

Clinical and laboratory evidence of exacerbations of hepatitis have occurred after discontinuation of lamivudine. See full prescribing information for EPIVIR (lamivudine). Patients should be closely monitored with both clinical and laboratory follow-up for at least several months after stopping treatment.

5.2 Hypersensitivity Reactions

Hypersensitivity reactions have been reported with the use of dolutegravir, a component of the combination and were characterized by rash, constitutional findings, and sometimes organ dysfunction, including liver injury. These events were reported in <1% of subjects receiving dolutegravir in Phase 3 clinical trials. Discontinue the combination immediately if signs or symptoms of hypersensitivity reactions develop (including, but not limited to, severe rash or rash accompanied by fever, general malaise, fatigue, muscle or joint aches, blisters or peeling of the skin, oral blisters or lesions, conjunctivitis, facial edema, hepatitis, eosinophilia, angioedema, difficulty breathing). Clinical status, including liver aminotransferases, should be monitored and appropriate therapy initiated. Delay in stopping treatment with the combination or other suspect agents after the onset of hypersensitivity may result in a life-threatening reaction.

5.3 Emergence of Lamivudine-Resistant HBV

Safety and efficacy of lamivudine have not been established for treatment of chronic hepatitis B in subjects dually infected with HIV-1 and HBV. Emergence of hepatitis B virus variants associated with resistance to lamivudine has been reported in HIV-1-infected subjects who have received lamivudine-containing antiretroviral regimens in the presence of concurrent infection with hepatitis B virus. See full prescribing information for EPIVIR-HBV (lamivudine).

5.4 Hepatotoxicity

Hepatic adverse events have been reported in patients receiving a dolutegravir-containing regimen [see Adverse Reactions (6.1)]. Patients with underlying hepatitis B or C may be at increased risk for worsening or development of transaminase elevations with use of the combination [see Adverse Reactions (6.1)]. In some cases, the elevations in transaminases were consistent with immune reconstitution syndrome or hepatitis B reactivation particularly in the setting where anti-hepatitis therapy was withdrawn. Cases of hepatic toxicity including elevated serum liver biochemistries, hepatitis, and acute liver failure have also been reported in patients receiving a dolutegravir-containing regimen who had no pre-existing hepatic disease or other identifiable risk factors. Drug-induced liver injury leading to liver transplant has been reported with TRIUMEQ (abacavir, dolutegravir, and lamivudine). Monitoring for hepatotoxicity is recommended.

5.5 Lactic Acidosis and Severe Hepatomegaly with Steatosis

Lactic acidosis and severe hepatomegaly with steatosis, including fatal cases, have been reported with the use of nucleoside analogues, including lamivudine (a component of the combination). A majority of these cases have been in women. Female sex and obesity may be risk factors for the development of lactic acidosis and severe hepatomegaly with steatosis in patients treated with antiretroviral nucleoside analogues. See full prescribing information for EPIVIR (lamivudine). Treatment with the combination should be suspended in any patient who develops clinical or laboratory findings suggestive of lactic acidosis or pronounced hepatotoxicity, which may include hepatomegaly and steatosis even in the absence of marked transaminase elevations.

5.6 Embryo-Fetal Toxicity

Preliminary data from an observational study showed that dolutegravir, a component of the combination, was associated with increased risk of neural tube defects when administered at the time of conception and in early pregnancy. As there is limited understanding of reported types of neural tube defects associated with dolutegravir use and because the date of conception may not be determined with precision, avoid use of the combination at the time of conception through the first trimester of pregnancy [see Use in Specific Populations (8.1)].

If there are plans to become pregnant or if pregnancy is confirmed within the first trimester while on the combination, if possible, switch to an alternative regimen.

Perform pregnancy testing before initiation of the combination in adolescents and adults of childbearing potential to exclude use of the combination during the first trimester of pregnancy [see Dosage and Administration (2.1)].

Advise adolescents and adults of childbearing potential to consistently use effective contraception [see Use in Specific Populations (8.1, 8.3)].

5.7 Risk of Adverse Reactions or Loss of Virologic Response Due to Drug Interactions

The concomitant use of the combination and other drugs may result in known or potentially significant drug interactions, some of which may lead to [see Contraindications (4), Drug Interactions (7.4)]:
Loss of therapeutic effect of the combination and possible development of resistance.
Possible clinically significant adverse reactions from greater exposures of concomitant drugs.

See Table 5 for steps to prevent or manage these possible and known significant drug interactions, including dosing recommendations. Consider the potential for drug interactions prior to and during therapy with the combination; review concomitant medications during therapy with the combination; and monitor for the adverse reactions associated with the concomitant drugs.

5.8 Immune Reconstitution Syndrome

Immune reconstitution syndrome has been reported in patients treated with combination antiretroviral therapy, including the combination. During the initial phase of combination antiretroviral treatment, patients whose immune systems respond may develop an inflammatory response to indolent or residual opportunistic infections (such as Mycobacterium avium infection, cytomegalovirus, Pneumocystis jirovecii pneumonia [PCP], or tuberculosis), which may necessitate further evaluation and treatment.

Autoimmune disorders (such as Graves' disease, polymyositis, and Guillain-Barrésyndrome) have also been reported to occur in the setting of immune reconstitution; however, the time to onset is more variable, and can occur many months after initiation of treatment.

6 Adverse Reactions

6.1 Clinical Trials Experience

Because clinical trials are conducted under widely varying conditions, adverse reaction rates observed in the clinical trials of a drug cannot be directly compared with rates in the clinical trials of another drug and may not reflect the rates observed in clinical practice.

The safety assessment of the combination in HIV-1-infected treatment-naive adult subjects with viral load ≤500,000 HIV-1 RNA copies/mL, is based on the pooled primary Week 48 analyses of data from 2 identical, multicenter, double-blind, controlled trials, GEMINI-1 and GEMINI-2. A total of 1,433 adult HIV-1-infected treatment-naive subjects were randomized to dolutegravir (TIVICAY) 50 mg plus lamivudine (EPIVIR) 300 mg, as a complete regimen once daily, or TIVICAY 50 mg plus fixed-dose combination tenofovir disoproxil fumarate (TDF)/emtricitabine (FTC) (TRUVADA), administered once daily.

The rates of adverse events leading to discontinuation in the pooled analysis were 2% of subjects in both treatment arms. The most common adverse events leading to discontinuation were psychiatric disorders: <1% of subjects in both treatment arms.

Adverse reactions (all grades) observed in at least 2% of subjects in either treatment arm of the Week 48 pooled analysis from GEMINI-1 and GEMINI-2 trials are provided in Table 2.

The adverse reactions observed for TIVICAY plus EPIVIR in the Week 48 analysis of the pooled data from GEMINI-1 and GEMINI-2 were generally consistent with the adverse reaction profiles and seventies for the individual components when administered with other antiretroviral agents.

TABLE 2
Adverse Reactions (All Grades) ≥2% Frequency in
either Treatment Arm in Treatment-Naive Subjects
in GEMINI-1 and GEMINI-2 (Week 48 Pooled Analysis)

| | TIVICAY plus | TIVICAY plus |
|---|---|---|
| | EPIVIR | TRUVADA |
| Adverse Reaction | (n = 716) | (n = 717) |
| Nervous System | | |
| Headache^{a} | 3% | 4% |
| Dizziness | 1% | 2% |
| Gastrointestinal | | |
| Diarrhea | 2% | 3% |
| Nausea | 2% | 5% |
| Psychiatric Disorders | | |
| Insomnia | 2% | 3% |

^{a}The only adverse reaction of ≥Grade 2 occurring in ≥1% of subjects treated with TIVICAY plus EPIVIR was headache (1%).

Less Common Adverse Reactions

The following adverse reactions occurred in <2% of subjects receiving dolutegravir plus lamivudine or are from studies described in the prescribing information of the individual components, TIVICAY (dolutegravir) and EPIVIR (lamivudine). Some events have been included because of their seriousness and assessment of potential causal relationship.

Blood and Lymphatic Systems Disorders: Anemia, neutropenia, thrombocytopenia.

Gastrointestinal Disorders: Abdominal discomfort, abdominal pain, flatulence, upper abdominal pain, vomiting.

General: Fatigue, fever, malaise.

Hepatobiliary Disorders: Hepatitis.

Immune System Disorders: Hypersensitivity, immune reconstitution syndrome.

Musculoskeletal Disorders: Myositis.

Nervous System Disorders: Somnolence.

Psychiatric Disorders: Abnormal dreams, depression. Suicidal ideation, attempt, behavior, or completion; these events were observed primarily in subjects with a pre-existing history of depression or other psychiatric illness.

Renal and Urinary Disorders: Renal impairment.

Skin and Subcutaneous Tissue Disorders: Pruritus, rash.

Laboratory Abnormalities

Selected laboratory abnormalities with a worsening grade from baseline and representing the worst-grade toxicity in ≥2% of subjects are presented in Table 3. The mean change from baseline observed for selected lipid values is presented in Table 4.

TABLE 3
Selected Laboratory Abnormalities (Grades 2 to 4; Week
48 Pooled Analyses) in GEMINI-1 and GEMINI-2 Trials

| | TIVICAY plus | TIVICAY plus |
|---|---|---|
| Laboratory Parameter | EPIVIR | TRUVADA |
| Preferred Term | (n = 716) | (n = 717) |
| ALT | | |
| Grade 2 (>2.5-5.0 × ULN) | 2% | 3% |
| Grade 3 to 4 (>5.0 × ULN) | 3% | 3% |
| AST | | |
| Grade 2 (>2.5-5.0 × ULN) | 3% | 3% |
| Grade 3 to 4 (>5.0 × ULN) | 2% | 3% |
| Total Bilirubin | | |
| Grade 2 (1.6-2.5 × ULN) | 1% | 2% |
| Grade 3 to 4 (>2.5 × ULN) | <1% | <1% |
| Creatine kinase | | |
| Grade 2 (6.0-9.9 × ULN) | 4% | 3% |
| Grade 3 to 4 (>10.0 × ULN) | 4% | 5% |
| Hyperglycemia | | |
| Grade 2 (126-250 mg/dL) | 7% | 4% |
| Grade 3 to 4 (>250 mg/dL) | <1% | <1% |
| Hypophosphatemia (Phosphate) | | |
| Grade 2 (1.4 to <2.0 mg/dL) | 7% | 8% |
| Grade 3 to 4 (<1.4 mg/dL) | <1% | <1% |
| Lipase | | |
| Grade 2 (>1.5-3.0 × ULN) | 5% | 5% |
| Grade 3 to 4 (>3.0 × ULN) | <1% | 3% |

ULN = Upper limit of normal.

TABLE 4
Mean Change from Baseline in Fasted Lipid Values (Week
48 Pooled Analyses^{a}) in GEMINI-1 and GEMINI-2 Trials

| | TIVICAY plus | TIVICAY plus |
|---|---|---|
| Laboratory Parameter | EPIVIR | TRUVADA |
| Preferred Term | (n = 716) | (n = 717) |
| Cholesterol (mg/dL) | 13.3 | −6.9 |
| HDL cholesterol (mg/dL) | 5.6 | 0.8 |
| LDL cholesterol (mg/dL) | 7.5 | −6.3 |
| Triglycerides (mg/dL) | 3.7 | −6.9 |
| Total cholesterol/HDL | −0.1 | −0.3 |
| cholesterol ratio | | |

^{a}Subjects on lipid-lowering agents at baseline are excluded (TIVICAY plus EPIVIR, n = 29; TIVICAY plus TRUVADA, n = 23). Lipid last observation carried forward (LOCF) data was used such that the last available fasted, on-treatment lipid value prior to the initiation of a lipid-lowering agent is used in place of future observed values. A total of 23 and 13 subjects receiving TIVICAY plus EPIVIR and TIVICAY plus TRUVADA, respectively, initiated lipid-lowering agents post-baseline.

Changes in Serum Creatinine: Dolutegravir has been shown to increase serum creatinine due to inhibition of tubular secretion of creatinine without affecting renal glomerular function [see Clinical Pharmacology (12.2)]. Increases in serum creatinine occurred within the first 4 weeks of treatment in both arms and remained stable through 48 weeks. A mean change from baseline of 0.116 mg/dL and 0.154 mg/dL was observed after 48 weeks of treatment with TIVICAY plus EPIVIR and TIVICAY plus TRUVADA, respectively. These changes are not considered to be clinically relevant.

6.2 Postmarketing Experience

In addition to adverse reactions reported from clinical trials, the following adverse reactions have been identified during postmarketing experience in patients receiving a dolutegravir- or lamivudine-containing regimen. Because these reactions are reported voluntarily from a population of uncertain size, it is not always possible to reliably estimate their frequency or establish a causal relationship to drug exposure: Redistribution/accumulation of body fat, Hyperglycemia, Weakness, Anemia (including pure red cell aplasia and severe anemias progressing on therapy), Lactic acidosis and hepatic steatosis [see Warnings and Precautions (5.5)], pancreatitis, posttreatment exacerbations of hepatitis B [see Warnings and Precautions (5.1)], Acute liver failure, hepatotoxicity, anaphylaxis, urticaria, weight increased, Arthralgia, CPK elevation, muscle weakness, myalgia, rhabdomyolysis, Paresthesia, peripheral neuropathy, anxiety, alopecia, and pruritus.

7 Drug Interactions

7.1 Concomitant Use with Other Antiretroviral Medicines

Because the combination is a complete regimen, coadministration with other antiretroviral medications for the treatment of HIV-1 infection is not recommended [see Indications and Usage (1)]. Information regarding potential drug-drug interactions with other antiretroviral medications is not provided [see Contraindications (4), Warnings and Precautions (5.7), Clinical Pharmacology (12.3)].

7.2 Potential for the Combination to Affect Other Drugs

Dolutegravir, a component of the combination, inhibits the renal organic cation transporters (OCT)2 and multidrug and toxin extrusion transporter (MATE)1, thus it may increase plasma concentrations of drugs eliminated via OCT2 or MATE1 such as dofetilide and metformin [see Contraindications (4), Drug Interactions (7.4), Clinical Pharmacology (12.3)].

7.3 Potential for Other Drugs to Affect the Components of the Combination

Dolutegravir is metabolized by uridine diphosphate (UDP)-glucuronosyl transferase (UGT)1A1 with some contribution from cytochrome P450 (CYP)3A. Dolutegravir is also a substrate of UGT1A3, UGT1A9, breast cancer resistance protein (BCRP), and P-glycoprotein (P-gp) in vitro. Drugs that induce those enzymes and transporters may decrease dolutegravir plasma concentrations and reduce the therapeutic effect of dolutegravir [see Drug Interactions (7.4), Clinical Pharmacology (12.3)]. Coadministration of dolutegravir and other drugs that inhibit these enzymes may increase dolutegravir plasma concentrations.

Coadministration of dolutegravir with polyvalent cation-containing products may lead to decreased absorption of dolutegravir [see Drug Interactions (7.4), Clinical Pharmacology (12.3)].

7.4 Established and Other Potentially Significant Drug Interactions

There were no drug-drug interaction trials conducted with the dolutegravir and lamivudine fixed-dose combination tablet.

Information regarding potential drug interactions with dolutegravir are provided in Table 5. These recommendations are based on either drug interaction trials or predicted interactions due to the expected magnitude of interaction and potential for serious adverse events or loss of efficacy [see Contraindications (4), Clinical Pharmacology (12.3)].

TABLE 5
Established and Other Potentially Significant Drug Interactions
for Dolutegravir: Alterations in Dose May Be Recommended Based
on Drug Interaction Trials or Predicted Interactions

| Concomitant Drug | Effect on | |
|---|---|---|
| Class: Drug Name | Concentration | Clinical Comment |
| Antiarrhythmic: | ↑Dofetilide | Coadministration is |
| Dofetilide | | contraindicated with the |
| | | combination [see |
| | | Contraindications (4)]. |
| Anticonvulsant: | ↓Dolutegravir | Adjust dolutegravir dose to 50 mg |
| Carbamazepine^{a} | | twice daily. An additional |
| | | dolutegravir 50-mg dose should be |
| | | taken, separated by 12 hours from |
| | | the combination. |
| Anticonvulsants: | ↓Dolutegravir | Avoid coadministration with the |
| Oxcarbazepine | | combination because there are |
| Phenytoin | | insufficient data to make dosing |
| Phenobarbital | | recommendations. |
| Antidiabetic: | ↑Metformin | With concomitant use, limit the |
| Metformin^{a} | | total daily dose of metformin to |
| | | 1,000 mg either when starting |
| | | metformin or the combination. |
| | | When starting or stopping the |
| | | combination, the metformin dose |
| | | may require an adjustment. |
| | | Monitoring of blood glucose when |
| | | initiating concomitant use and |
| | | after withdrawal of the |
| | | combination is recommended. |
| Antimycobacterial: | ↓Dolutegravir | Adjust dolutegravir dose to 50 mg |
| Rifampin^{a} | | twice daily. An additional 50-mg |
| | | dose of dolutegravir should be |
| | | taken, separated by 12 hours from |
| | | the combination. |
| Herbal Product: | ↓Dolutegravir | Avoid coadministration with the |
| St. John's wort | | combination because there are |
| (Hypericum | | insufficient data to make dosing |
| perforatum) | | recommendations. |
| Medications | ↓Dolutegravir | Administer the combination 2 |
| containing | | hours before or 6 hours after |
| polyvalent | | taking medications containing |
| cations | | polyvalent cations. |
| (e.g., Mg or Al): | | |
| Cation-containing | | |
| antacids^{a} or | | |
| laxatives | | |
| Sucralfate | | |
| Buffered | | |
| medications | | |
| Oral calcium and | ↓Dolutegravir | Administer the combination 2 |
| iron supplements, | | hours before or 6 hours after |
| including | | taking supplements containing |
| multivitamins | | calcium or iron. Alternatively, the |
| containing | | combination and supplements |
| calcium or | | containing calcium or iron can be |
| iron^{a} | | taken together with food. |

↑= Increase.
↓= Decrease.
^{a}See Clinical Pharmacology (12.3) Table 8 or Table 9 for magnitude of interaction.

Sorbitol

Lamivudine: Coadministration of single doses of lamivudine and sorbitol solutions resulted in a sorbitol dose-dependent reduction in lamivudine exposures. When possible, avoid use of sorbitol-containing medicines with lamivudine-containing medicines [see Clinical Pharmacology (12.3)].

8 Use in Specific Populations

8.1 Pregnancy

Pregnancy Exposure Registry

There is a pregnancy exposure registry that monitors pregnancy outcomes in individuals exposed to the combination during pregnancy. Healthcare providers are encouraged to register patients by calling the Antiretroviral Pregnancy Registry (APR) at 1-800-258-4263.

Risk Summary

Preliminary data from an observational study has identified a possible increased risk of neural tube defects when dolutegravir, a component of the combination, is administered at the time of conception compared with non-dolutegravir-containing antiretroviral regimens. As defects related to closure of the neural tube occur from conception through the first 6 weeks of gestation, embryos exposed to dolutegravir from the time of conception through the first 6 weeks of gestation are at potential risk. In addition, 2 of the 4 birth defects (encephalocele and iniencephaly), which have been observed with dolutegravir use, although often termed neural tube defects, may occur post-neural tube closure, the time period of which may be later than 6 weeks of gestation, but within the first trimester. Due to the limited understanding of the types of reported neural tube defects associated with dolutegravir use and because the date of conception may not be determined with precision, avoid use of the combination at the time of conception through the first trimester of pregnancy. No neural tube defects have been reported in infants born to mothers who have started dolutegravir after the first trimester of pregnancy (see Data).

If there are plans to become pregnant or if pregnancy is confirmed while on the combination during the first trimester, if possible, switch to an alternative regimen. Advise pregnant adolescents and adults of the potential risk to the embryo exposed to the combination from the time of conception through the first trimester of pregnancy.

There are insufficient human data on the use of the combination during pregnancy to definitively assess a drug-associated risk for birth defects and miscarriage. The background risk for major birth defects for the indicated population is unknown. In the U.S. general population, the estimated background rate for major birth defects and miscarriage in clinically recognized pregnancies is 2% to 4% and 15% to 20%, respectively.

In animal reproduction studies, no evidence of adverse developmental outcomes was observed with dolutegravir at systemic exposures (AUC) less than (rabbits) and 38 times (rats) the exposure in humans at the recommended human dose (RHD) (see Data). In the rat pre/post-natal developmental studies, maternal systemic exposure (AUC) to dolutegravir was approximately 32 times the exposure in humans at the RHD. Oral administration of lamivudine to pregnant rabbits during organogenesis resulted in embryolethality at systemic exposure (AUC) similar to the RHD; however, no adverse developmental effects were observed with oral administration of lamivudine to pregnant rats during organogenesis at plasma concentrations (Cₘₐₓ) 32 times the RHD (see Data).

Data

Human Data: Dolutegravir: As of May 2018, in an ongoing birth outcome surveillance study in Botswana, there have been 4 cases of neural tube defects reported out of 426 births (0.94%) to mothers who were exposed to dolutegravir-containing regimens at the time of conception. In comparison, the neural tube defect prevalence rates were 0.12% (14/11,300) in the non-dolutegravir arm and 0.09% (61/66,057) in the HIV-uninfected arm. Four cases reported with dolutegravir included one case each of encephalocele, anencephaly, myelomeningocele, and iniencephaly. No infant born to a woman who started dolutegravir during pregnancy had a neural tube defect (n=2,812).

Data analyzed to date from other sources including the APR, clinical trials, and postmarketing data are insufficient to address the risk of neural tube defects with dolutegravir.

Lamivudine: Based on prospective reports to the APR of over 11,000 exposures to lamivudine during pregnancy resulting in live births (including over 4,500 exposed in the first trimester), there was no difference between the overall risk of birth defects for lamivudine compared with the background birth defect rate of 2.7% in the U.S. reference population of the MACDP. The prevalence of defects in live births was 3.1% (95% CI: 2.6% to 3.6%) following first trimester exposure to lamivudine-containing regimens and 2.8% (95% CI: 2.5%, 3.3%) following second/third trimester exposure to lamivudine-containing regimens.

Lamivudine pharmacokinetics were studied in pregnant women during 2 clinical trials conducted in South Africa. The trials assessed pharmacokinetics in 16 women at 36 weeks' gestation using 150 mg lamivudine twice daily with zidovudine, 10 women at 38 weeks' gestation using 150 mg lamivudine twice daily with zidovudine, and 10 women at 38 weeks' gestation using lamivudine 300 mg twice daily without other antiretrovirals. These trials were not designed or powered to provide efficacy information. Lamivudine concentrations were generally similar in maternal, neonatal, and umbilical cord serum samples. In a subset of subjects, amniotic fluid specimens were collected following natural rupture of membranes and confirmed that lamivudine crosses the placenta in humans. Based on limited data at delivery, median (range) amniotic fluid concentrations of lamivudine were 3.9-fold (1.2- to 12.8-fold) greater compared with paired maternal serum concentration (n=8).

Animal Data: Dolutegravir: Dolutegravir was administered orally at up to 1,000 mg/kg daily to pregnant rats and rabbits on gestation Days 6 to 17 and 6 to 18, respectively, and also to rats on gestation Day 6 to lactation/post-partum Day 20. No adverse effects on embryo-fetal (rats and rabbits) or pre/post-natal (rats) development were observed up to the highest dose tested. During organogenesis, systemic exposures (AUC) to dolutegravir in rabbits were less than the exposure in humans at the RHD and in rats were approximately 38 times the exposure in humans at the RHD. In the rat pre/post-natal development study, decreased body weight of the developing offspring was observed during lactation at a maternally toxic dose (approximately 32 times human exposure at the RHD).

Lamivudine: Lamivudine was administered orally to pregnant rats (at 90, 600, and 4,000 mg/kg/day) and rabbits (at 90, 300 and 1,000 mg/kg/day and at 15, 40, and 90 mg/kg/day) during organogenesis (on gestation Days 7 through 16 [rat] and 8 through 20 [rabbit]). No evidence of fetal malformations due to lamivudine was observed in rats and rabbits at doses producing plasma concentrations (Cₘₐₓ) approximately 32 times higher than human exposure at the RHD. Evidence of early embryolethality was seen in the rabbit at systemic exposures (AUC) similar to those observed in humans, but there was no indication of this effect in the rat at plasma concentrations (Cₘₐₓ) 32 times higher than human exposure at the RHD. Studies in pregnant rats showed that lamivudine is transferred to the fetus through the placenta. In the fertility/pre- and postnatal development study in rats, lamivudine was administered orally at doses of 180, 900, and 4,000 mg/kg/day (from prior to mating through postnatal Day 20). In the study, development of the offspring, including fertility and reproductive performance, was not affected by the maternal administration of lamivudine.

8.2 Lactation

Risk Summary

The Centers for Disease Control and Prevention recommends that HIV-1-infected mothers in the United States not breastfeed their infants to avoid risking postnatal transmission of HIV-1 infection.

Lamivudine, a component of the combination, is present in human milk It is not known whether dolutegravir, a component of the combination, is present in human milk. When administered to lactating rats, dolutegravir was present in milk (see Data). There is no information on the effects of the combination or the components of the combination (dolutegravir and lamivudine) on the breastfed infant or the effects of the drugs on milk production.

Because of the potential for (1) HIV-1 transmission (in HIV-negative infants), (2) developing viral resistance (in HIV-positive infants), and (3) adverse reactions in a breastfed infant similar to those seen in adults, instruct mothers not to breastfeed if they are receiving the combination.

Data

Animal Data: Dolutegravir was the primary drug-related component excreted into the milk of lactating rats following a single oral dose of 50 mg/kg on lactation Day 10, with milk concentrations of up to approximately 1.3 times that of maternal plasma concentrations observed 8 hours postdose.

8.3 Females and Males of Reproductive Potential

Pregnancy Testing

Perform pregnancy testing in adolescents and adults of childbearing potential before initiation of the combination.

Contraception

Adolescents and adults of childbearing potential should avoid use of the combination at the time of conception through the first trimester of pregnancy because of the potential risk of neural tube defects [see Use in Specific Populations (8.1)].

Advise adolescents and adults of childbearing potential who are taking the combination to consistently use effective contraception.

8.4 Pediatric Use

The safety and efficacy of the combination have not been established in pediatric patients.

8.5 Geriatric Use

Clinical trials of the combination did not include sufficient numbers of subjects aged 65 and over to determine whether they respond differently from younger subjects. In general, caution should be exercised in the administration of the combination in elderly patients reflecting the greater frequency of decreased hepatic, renal, or cardiac function, and of concomitant disease or other drug therapy [see Clinical Pharmacology (12.3)].

8.6 Renal Impairment

The combination is not recommended for patients with creatinine clearance <50 mL/min because the combination is a fixed-dose combination and the dosage of the individual components cannot be adjusted. If a dose reduction of lamivudine, a component of the combination, is required for patients with creatinine clearance <50 mL/min, then the individual components should be used [see Clinical Pharmacology (12.3)].

8.7 Hepatic Impairment

No dosage adjustment of the combination is necessary in patients with mild or moderate (Child-Pugh Score A or B) hepatic impairment. Dolutegravir has not been studied in patients with severe hepatic impairment (Child-Pugh Score C); therefore, the combination is not recommended for patients with severe hepatic impairment [see Clinical Pharmacology (12.3)].

10 Overdosage

There is no known specific treatment for overdose with the combination. If overdose occurs, the patient should be monitored and standard supportive treatment applied as required.

Dolutegravir

As dolutegravir is highly bound to plasma proteins, it is unlikely that it will be significantly removed by dialysis.

Lamivudine

Because a negligible amount of lamivudine was removed via (4-hour) hemodialysis, continuous ambulatory peritoneal dialysis, and automated peritoneal dialysis, it is not known if continuous hemodialysis would provide clinical benefit in a lamivudine overdose event.

11 Description

The combination is a fixed-dose combination tablet containing dolutegravir (as dolutegravir sodium), an INSTI, and lamivudine (also known as 3TC), a nucleoside analogue.

The combination tablets are for oral administration. Each film-coated tablet contains the active ingredients 50 mg of dolutegravir (equivalent to 52.6 mg dolutegravir sodium) and 300 mg of lamivudine and the inactive ingredients magnesium stearate, mannitol, microcrystalline cellulose, povidone K29/32, sodium starch glycolate, sodium stearyl fumarate. The tablet film-coating contains the inactive ingredients hypromellose, polyethylene glycol, titanium dioxide.

Dolutegravir

The chemical name of dolutegravir sodium is sodium (4R,12aS)-9-{[(2,4-difluorophenyl)methyl]carbamoyl}-4-methyl-6,8-dioxo-3,4,6,8,12,12a-hexahydro-2H-pyrido[1′, 2′:4,5]pyrazino[2,1-b][1,3]oxazin-7-olate. The empirical formula is C₂₀H₁₈F₂N₃NaO₅ and the molecular weight is 441.36 g/mol. It has the following structural formula:

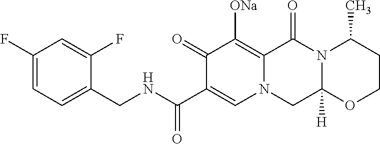

Dolutegravir sodium is a white to light yellow powder and is slightly soluble in water.

Lamivudine

The chemical name of lamivudine is (2R,cis)-4-amino-1-(2-hydroxymethyl-1,3-oxathiolan-5-yl)-(1H)-pyrimidin-2-one. Lamivudine is the (−)enantiomer of a dideoxy analogue of cytidine. Lamivudine has also been referred to as (−)2′,3′-dideoxy, 3′-thiacytidine. It has a molecular formula of C₈H₁₁N₃O₃S and a molecular weight of 229.3 g/mol. It has the following structural formula:

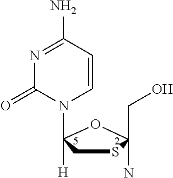

Lamivudine is a white to off-white crystalline solid and is soluble in water.

12 Clinical Pharmacology

12.1 Mechanism of Action

The combination is a fixed-dose combination of the HIV-1 antiretroviral agents, dolutegravir and lamivudine [see Microbiology (12.4)].

12.2 Pharmacodynamics

Cardiac Electrophysiology

The effect of the combination on the QT interval has not been studied.

A thorough QT trial has been conducted for dolutegravir. The effect of lamivudine on the QT interval has not been evaluated.

In a randomized, placebo-controlled, cross-over trial, 42 healthy subjects received single-dose oral administrations of placebo, dolutegravir 250-mg suspension (exposures approximately 3-fold that of the 50-mg once-daily dose at steady state), and moxifloxacin 400 mg (active control) in random sequence. After baseline and placebo adjustment, the maximum mean QTc change based on Fridericia correction method (QTcF) for dolutegravir was 2.4 msec (1-sided 95% upper CI: 4.9 msec). Dolutegravir did not prolong the QTc interval over 24 hours postdose.

Effects on Renal Function

The effect of dolutegravir on renal function was evaluated in an open-label, randomized, 3-arm, parallel, placebo-controlled trial in healthy subjects (n=37) who received dolutegravir 50 mg once daily (n=12), dolutegravir 50 mg twice daily (n=13), or placebo once daily (n=12) for 14 days. A decrease in creatinine clearance, as determined by 24-hour urine collection, was observed with both doses of dolutegravir after 14 days of treatment in subjects who received 50 mg once daily (9% decrease) and 50 mg twice daily (13% decrease). Neither dose of dolutegravir had a significant effect on the actual glomerular filtration rate (determined by the clearance of probe drug, iohexol) or effective renal plasma flow (determined by the clearance of probe drug, para-amino hippurate) compared with the placebo.

12.3 Pharmacokinetics

Absorption, Distribution, Metabolism, and Excretion

The pharmacokinetic (PK) properties of the components of the combination are provided in Table 6. The multiple-dose pharmacokinetic parameters are provided in Table 7.

TABLE 6
Pharmacokinetic Properties of the Components of the combination

| | | |
|---|---|---|
| Pharmacokinetic Parameters | Dolutegravir | Lamivudine |

Absorption

| | | |
|---|---|---|
| Tₘₐₓ (h), median | 2.5 | 1 |
| Effect of high-fat meal | AUC Ratio | AUC Ratio |
| (relative to fasting)^{a} | 1.33 (1.18, 1.48) | 0.91 (0.87, 0.96) |

Distribution

| | | |
|---|---|---|
| % Bound to human | ~99 | 36^{b} |
| plasma proteins | | |
| Source of protein | in vitro | in vitro |
| binding data | | |
| Blood-to-plasma ratio | 0.44-0.54 | 1.1-1.2 |

Metabolism

| | | |
|---|---|---|
| Metabolic pathways | UGT1A1 | Not significantly |
| | CYP3A (minor) | metabolized |

Elimination

| | | |
|---|---|---|
| Major route of | Metabolism | Renal, by the OCT |
| elimination | | system |
| t_{1/2} (h) | ~14 | 18-19 |
| % of dose excreted as | <1^{b} | ~70^{c} |
| unchanged drug in the | | |
| urine | | |
| % of dose excreted as | 31^{b} | — |
| total ¹⁴C in urine^{b} | | |
| % of dose excreted as | 64 (53)^{b} | — |
| total ¹⁴C (unchanged | | |
| drug) in feces^{b} | | |

^{a}Geometric mean ratio (fed/fasted) in PK parameters and (90% confidence interval). High-calorie/high-fat meal = ~900 kcal, 56% fat.
^{b}Based on single-dose, mass balance study of [¹⁴C] dolutegravir.
^{c}Based on 24-hour urine collection obtained after oral or IV administration (NUCB1001).

TABLE 7
Multiple-Dose Pharmacokinetic Properties
of the Components of the combination

| Parameter Mean (CV %) | Dolutegravir^{a} | Lamivudine^{b} | |
|---|---|---|---|
| Cₘₐₓ (mcg/mL) | 3.67 (20%) | 2.04 | (26%) |
| AUCₜₐᵤ (mcg/h/mL) | 53.6 (27%) | 8.87 | (21%) |
| C_{trough} (mcg/mL) | 1.11 (46%) | 0.042 | (38%) |

^{a}Based on population pharmacokinetic analyses using pooled data from antiretroviral (ART) treatment-naive adults receiving 50 mg dolutegravir once daily.
^{b}Based on pharmacokinetic data from healthy subjects (n = 60) administered 300 mg once daily.

Specific Populations

Pediatric Patients: The pharmacokinetics of the combination has not been studied in pediatric subjects [see Use in Specific Populations (8.4)].

Geriatric Patients: Population pharmacokinetic analyses indicated age had no clinically relevant effect on the pharmacokinetics of dolutegravir. Pharmacokinetic data for dolutegravir and lamivudine in subjects aged 65 years and older are limited [see Use in Specific Populations (8.5)].

Patients with Renal Impairment: The effect of renal impairment on the combination of dolutegravir and lamivudine has not been evaluated. See full prescribing information for TIVICAY (dolutegravir) and EPIVIR (lamivudine).

Patients with Hepatic Impairment: The effect of hepatic impairment on the combination of dolutegravir and lamivudine has not been evaluated. See full prescribing information for TIVICAY (dolutegravir) and EPIVIR (lamivudine).

Gender and Race: Population pharmacokinetic analyses from studies with the individual components revealed that gender and race had no clinically relevant effect on the pharmacokinetics of dolutegravir or lamivudine.

Drug Interaction Studies

Drug interaction trials described were conducted with dolutegravir and/or lamivudine as single entities; no drug interaction trials have been conducted using the combination of dolutegravir and lamivudine. No clinically significant drug interactions are expected between dolutegravir and lamivudine.

In vitro, dolutegravir did not inhibit (IC₅₀>50 microM) the following: CYP1A2, CYP2A6, CYP2B6, CYP2C8, CYP2C9, CYP2C19, CYP2D6, CYP3A, UGT1A1, UGT2B7, P-gp, BCRP, bile salt export pump (BSEP), organic anion transporter polypeptide (OATP)1B1, OATP1B3, OCT1, multidrug resistance protein (MRP)2, or MRP4. In vitro, dolutegravir did not induce CYP1A2, CYP2B6, or CYP3A4.

In vitro, dolutegravir inhibited the renal OCT2 (IC₅₀=1.93 microM) and MATE1 (IC₅₀=6.34 microM). In vivo, dolutegravir inhibits tubular secretion of creatinine by inhibiting OCT2 and potentially MATE1. Dolutegravir may increase plasma concentrations of drugs eliminated via OCT2 or MATE1 such as dofetilide and metformin [see Contraindications (4), Drug Interactions (7.4)].

In vitro, dolutegravir inhibited the basolateral renal transporters, organic anion transporter (OAT)1 (IC₅₀=2.12 microM) and OAT3 (IC₅₀=1.97 microM). However, in vivo, dolutegravir did not alter the plasma concentrations of tenofovir or para-amino hippurate, substrates of OAT1 and OAT3.

Dolutegravir is metabolized by UGT1A1 with some contribution from CYP3A. Dolutegravir is also a substrate of UGT1A3, UGT1A9, BCRP, and P-gp in vitro. In vitro, dolutegravir was not a substrate of OATP1B1 or OATP1B3.

Dosing recommendations as a result of established and other potentially significant drug-drug interactions with the individual components of the combination are provided in Section 7.4 [see Drug Interactions (7)].

TABLE 8
Summary of Effect of Dolutegravir on the Pharmacokinetics of Coadministered Drugs

| | | | Geometric Mean Ratio (90% CI) of | | |
|---|---|---|---|---|---|
| | | | Pharmacokinetic Parameters of | | |
| | | | Coadministered Drug with/without | | |
| Coadministered | | | Dolutegravir | | |
| Drug(s) | Dose of | | No Effect = 1.00 | | |
| and Dose(s) | Dolutegravir | n | Cₘₐₓ | AUC | C_{τ} or C₂₄ |
| Daclatasvir | 50 mg | 12 | 1.03 | 0.98 | 1.06 |
| 60 mg once daily | once daily | | (0.84 to 1.25) | (0.83 to 1.15) | (0.88 to 1.29) |
| Ethinyl estradiol | 50 mg | 15 | 0.99 | 1.03 | 1.02 |
| 0.035 mg | twice daily | | (0.91 to 1.08) | (0.96 to 1.11) | (0.93 to 1.11) |
| Grazoprevir | 50 mg | 12 | 0.64 | 0.81 | 0.86 |
| 200 mg once daily | single dose | | (0.44, 0.93) | (0.67, 0.97) | (0.79, 0.93) |
| Metformin | 50 mg | 15^{a} | 1.66 | 1.79 | — |
| 500 mg twice daily | once daily | | (1.53 to 1.81) | (1.65 to 1.93) | |
| Metformin | 50 mg | 15^{a} | 2.11 | 2.45 | — |
| 500 mg twice daily | twice daily | | (1.91 to 2.33) | (2.25 to 2.66) | |
| Methadone | 50 mg | 11 | 1.00 | 0.98 | 0.99 |
| 16 to 150 mg | twice daily | | (0.94 to 1.06) | (0.91 to 1.06) | (0.91 to 1.07) |
| Midazolam | 25 mg | 10 | — | 0.95 | — |
| 3 mg | once daily | | | (0.79 to 1.15) | |
| Norelgestromin | 50 mg | 15 | 0.89 | 0.98 | 0.93 |
| 0.25 mg | twice daily | | (0.82 to 0.97) | (0.91 to 1.04) | (0.85 to 1.03) |
| Sofosbuvir | 50 mg | 24 | 0.88 | 0.92 | NA |
| 400 mg once daily | once daily | | (0.80, 0.98) | (0.85, 0.99) | |
| Metabolite (GS-331007) | | | 1.01 | 0.99 | 0.99 |
| | | | (0.93, 1.10) | (0.97, 1.01) | (0.97, 1.01) |
| Velpatasvir | 50 mg | 24 | 0.94 | 0.91 | 0.88 |
| 100 mg once daily | once daily | | (0.86, 1.02) | (0.84, 0.98) | (0.82, 0.94) |

^{a}The number of subjects represents the maximum number of subjects that were evaluated.

TABLE 9
Summary of Effect of Coadministered Drugs on the Pharmacokinetics of Dolutegravir

| | | | Geometric Mean Ratio (90% CI) of | | |
|---|---|---|---|---|---|
| | | | Dolutegravir Pharmacokinetic Parameters | | |
| Coadministered | | | with/without Coadministered Drugs | | |
| Drug(s) | Dose of | | No Effect = 1.00 | | |
| and Dose(s) | Dolutegravir | n | Cₘₐₓ | AUC | C_{τ} or C₂₄ |
| Antacid (MAALOX) | 50 mg | 16 | 0.28 | 0.26 | 0.26 |
| simultaneous | single dose | | (0.23 to 0.33) | (0.22 to 0.32) | (0.21 to 0.31) |
| administration | | | | | |
| Antacid (MAALOX) | 50 mg | 16 | 0.82 | 0.74 | 0.70 |
| 2 h after dolutegravir | single dose | | (0.69 to 0.98) | (0.62 to 0.90) | (0.58 to 0.85) |
| Calcium carbonate | 50 mg | 12 | 0.63 | 0.61 | 0.61 |
| 1,200 mg | single dose | | (0.50 to 0.81) | (0.47 to 0.80) | (0.47 to 0.80) |
| simultaneous | | | | | |
| administration | | | | | |
| (fasted) | | | | | |
| Calcium carbonate | 50 mg | 11 | 1.07 | 1.09 | 1.08 |
| 1,200 mg | single dose | | (0.83 to 1.38) | (0.84 to 1.43) | (0.81 to 1.42) |
| simultaneous | | | | | |
| administration | | | | | |
| (fed) | | | | | |
| Calcium carbonate | 50 mg | 11 | 1.00 | 0.94 | 0.90 |
| 1,200 mg | single dose | | (0.78 to 1.29) | (0.72 to 1.23) | (0.68 to 1.19) |
| 2 h after dolutegravir | | | | | |
| Carbamazepine | 50 mg | 16^{a} | 0.67 | 0.51 | 0.27 |
| 300 mg twice daily | once daily | | (0.61 to 0.73) | (0.48 to 0.55) | (0.24 to 0.31) |
| Daclatasvir | 50 mg | 12 | 1.29 | 1.33 | 1.45 |
| 60 mg once daily | once daily | | (1.07 to 1.57) | (1.11 to 1.59) | (1.25 to 1.68) |
| Ferrous fumarate | 50 mg | 11 | 0.43 | 0.46 | 0.44 |
| 324 mg | single dose | | (0.35 to 0.52) | (0.38 to 0.56) | (0.36 to 0.54) |
| simultaneous | | | | | |
| administration | | | | | |
| (fasted) | | | | | |
| Ferrous fumarate | 50 mg | 11 | 1.03 | 0.98 | 1.00 |
| 324 mg | single dose | | (0.84 to 1.26) | (0.81 to 1.20) | (0.81 to 1.23) |
| simultaneous | | | | | |
| administration | | | | | |
| (fed) | | | | | |
| Ferrous fumarate | 50 mg | 10 | 0.99 | 0.95 | 0.92 |
| 324 mg | single dose | | (0.81 to 1.21) | (0.77 to 1.15) | (0.74 to 1.13) |
| 2 h after dolutegravir | | | | | |
| Multivitamin | 50 mg | 16 | 0.65 | 0.67 | 0.68 |
| (One-A-Day) | single dose | | (0.54 to 0.77) | (0.55 to 0.81) | (0.56 to 0.82) |
| simultaneous | | | | | |
| administration | | | | | |
| Omeprazole | 50 mg | 12 | 0.92 | 0.97 | 0.95 |
| 40 mg once daily | single dose | | (0.75 to 1.11) | (0.78 to 1.20) | (0.75 to 1.21) |
| Prednisone | 50 mg | 12 | 1.06 | 1.11 | 1.17 |
| 60 mg once daily | once daily | | (0.99 to 1.14) | (1.03 to 1.20) | (1.06 to 1.28) |
| with taper | | | | | |
| Rifampin^{b} | 50 mg | 11 | 0.57 | 0.46 | 0.28 |
| 600 mg once daily | twice daily | | (0.49 to 0.65) | (0.38 to 0.55) | (0.23 to 0.34) |
| Rifampin^{c} | 50 mg | 11 | 1.18 | 1.33 | 1.22 |
| 600 mg once daily | twice daily | | (1.03 to 1.37) | (1.15 to 1.53) | (1.01 to 1.48) |
| Rifabutin | 50 mg | 9 | 1.16 | 0.95 | 0.70 |
| 300 mg once daily | once daily | | (0.98 to 1.37) | (0.82 to 1.10) | (0.57 to 0.87) |

^{a}The number of subjects represents the maximum number of subjects that were evaluated.
^{b}Comparison is rifampin taken with dolutegravir 50 mg twice daily compared with dolutegravir 50 mg twice daily.
^{c}Comparison is rifampin taken with dolutegravir 50 mg twice daily compared with dolutegravir 50 mg once daily.

Lamivudine: The drug interactions described are based on trials conducted with lamivudine as a single entity.

Effect of Lamivudine on the Pharmacokinetics of Other Agents: Based on in vitro study results, lamivudine at therapeutic drug exposures is not expected to affect the pharmacokinetics of drugs that are substrates of the following transporters: OATP1B1/3, BCRP, P- , MATE1, MATE2-K, OCT1, OCT2, or OCT3.

Effect of Other Agents on the Pharmacokinetics of Lamivudine: Lamivudine is a substrate of MATE1, MATE2-K, and OCT2 in vitro. Trimethoprim (an inhibitor of these drug transporters) has been shown to increase lamivudine plasma concentrations. This interaction is not considered clinically significant as no dose adjustment of lamivudine is needed.

Lamivudine is a substrate of P-gp and BCRP; however, considering its absolute bioavailability (87%), it is unlikely that these transporters play a significant role in the absorption of lamivudine. Therefore, coadministration of drugs that are inhibitors of these efflux transporters is unlikely to affect the disposition and elimination of lamivudine.

Interferon Alfa: There was no significant pharmacokinetic interaction between lamivudine and interferon alfa in a trial of 19 healthy male subjects.

Ribavirin: In vitro data indicate ribavirin reduces phosphorylation of lamivudine, stavudine, and zidovudine. However, no pharmacokinetic (e.g., plasma concentrations or intracellular triphosphorylated active metabolite concentrations) or pharmacodynamic (e.g., loss of HIV-1/HCV virologic suppression) interaction was observed when ribavirin and lamivudine (n=18), stavudine (n=10), or zidovudine (n=6) were coadministered as part of a multi-drug regimen to HIV-1/HCV co-infected subjects.

Sorbitol (Excipient): Lamivudine and sorbitol solutions were coadministered to 16 healthy adult subjects in an open-label, randomized sequence, 4-period, crossover trial. Each subject received a single 300-mg dose of lamivudine oral solution alone or coadministered with a single dose of 3.2 grams, 10.2 grams, or 13.4 grams of sorbitol in solution. Coadministration of lamivudine with sorbitol resulted in dose-dependent decreases of 20%, 39%, and 44% in the AUC₍₀₋₂₄₎; 14%, 32%, and 36% in the AUC_{(∞)}; and 28%, 52%, and 55% in the Cₘₐₓ of lamivudine, respectively.

The effects of other coadministered drugs on lamivudine are provided in Table 10.

TABLE 10
Effect of Coadministered Drugs on Lamivudine

| | | | Concentrations of | | Concentration of |
|---|---|---|---|---|---|
| Coadministered | | | Lamivudine | | Coadministered |
| Drug and Dose | Drug and Dose | n | AUC | Variability | Drug |
| Trimethoprim 160 mg/ | Lamivudine | 14 | ↑43% | 90% CI: | ↔ |
| Sulfamethoxazole | Single 300 mg | | | 32% to 55% | |
| 800 mg daily × 5 days | | | | | |

↑= Increase; ↔ = No significant change; AUC = Area under the concentration versus time curve; CI = Confidence interval; NA = Not applicable.

12.4 Microbiology

Mechanism of Action

Dolutegravir: Dolutegravir inhibits HIV integrase by binding to the integrase active site and blocking the strand transfer step of retroviral DNA integration which is essential for the HIV replication cycle. Strand transfer biochemical assays using purified recombinant HIV-1 integrase and pre-processed substrate DNA resulted in IC₅₀ values of 2.7 nM and 12.6 nM.

Lamivudine: Lamivudine is a synthetic nucleoside analogue. Intracellularly lamivudine is phosphorylated to its active 5′-triphosphate metabolite, lamivudine triphosphate (3TC-TP). The principal mode of action of 3TC-TP is inhibition of reverse transcriptase (RT) via DNA chain termination after incorporation of the nucleotide analogue.

Antiviral Activity in Cell Culture

Dolutegravir: Dolutegravir exhibited antiviral activity against laboratory strains of wild-type HIV-1 with mean concentrations of drug necessary to effect viral replication by 50 percent (EC₅₀) values of 0.5 nM (0.21 ng/mL) to 2.1 nM (0.85 ng/mL) in peripheral blood mononuclear cells (PBMCs) and MT-4 cells.

Dolutegravir exhibited antiviral activity against 13 clinically diverse clade B isolates with a mean EC₅₀ value of 0.52 nM in a viral integrase susceptibility assay using the integrase coding region from clinical isolates. Dolutegravir demonstrated antiviral activity in cell culture against a panel of HIV-1 clinical isolates (3 in each group of M [clades A-G], and 3 in group O) with EC₅₀ values ranging from 0.02 nM to 2.14 nM for HIV-1. Dolutegravir EC₅₀ values against three HIV-2 clinical isolates in PBMC assays ranged from 0.09 nM to 0.61 nM.

Lamivudine: The antiviral activity of lamivudine against HIV-1 was assessed in a number of cell lines including monocytes and PBMCs using standard susceptibility assays. EC₅₀ values were in the range of 0.003 to 15 microM (1 microM=0.23 mcg/mL). The EC₅₀ values of lamivudine against different HIV-1 clades (A-G) and group O viruses ranged from 0.001 to 0.120 microM, and against HIV-2 isolates from 0.003 to 0.120 microM in PBMCs.

Antiviral Activity in Combination with Other Antiviral Agents

Neither dolutegravir nor lamivudine were antagonistic to all tested anti-HIV agents. See full prescribing information for TIVICAY (dolutegravir) and EPIVIR (lamivudine).

Resistance

Cell Culture:

Dolutegravir: Dolutegravir-resistant viruses were selected in cell culture starting from different wild-type HIV-1 strains and clades Amino acid substitutions emerged in different passages; G118R emergence conferred decreased susceptibility to dolutegravir of 10-fold, while substitutions E92Q, S153F or Y, G193E or R263K conferred decreased susceptibility to dolutegravir of up to 4-fold.

Lamivudine: HIV-1 resistance to lamivudine involves the development of a M184I or M184V amino acid change close to the active site of the viral RT. This variant arises both in vitro and in HIV-1 infected patients treated with lamivudine-containing antiretroviral therapy. M184V mutants display greatly reduced susceptibility to lamivudine and show diminished viral replicative capacity in vitro.

Clinical Subjects: None of the 6 subjects in the dolutegravir plus lamivudine group or the 4 subjects in the dolutegravir plus TDF/FTC group that met the protocol-defined confirmed virologic withdrawal (CVW) criteria across the pooled GEMINI-1 and GEMINI-2 studies through Week 48 had emergent INSTI or NRTI resistance substitutions.

Cross-Resistance

Dolutegravir: The susceptibility of dolutegravir was tested against 60 INSTI-resistant site-directed mutant HIV-1 viruses (28 with single substitutions and 32 with 2 or more substitutions). The single INSTI-resistance substitutions T66K, I151L, and S153Y conferred a >2-fold decrease in dolutegravir susceptibility (range: 2.3-fold to 3.6-fold from reference). Combinations of multiple substitutions T66K/L74M; E92Q/N155H; G140C/Q148R; G140S/Q148H, R or K; Q148R/N155H; T97A/G140S/Q148, and substitutions at E138/G140/Q148 showed a >2-fold decrease in dolutegravir susceptibility (range: 2.5-fold to 21-fold from reference).

Lamivudine: Cross-resistance conferred by the M184V RT is limited within the nucleoside inhibitor class of antiretroviral agents. Zidovudine maintains its antiretroviral activities against lamivudine-resistant HIV-1. Abacavir and tenofovir maintain antiretroviral activity against lamivudine-resistant HIV-1 harboring only the M184V substitution. Cross-resistance is expected with emtricitabine which also selects the M184V substitution and abacavir which selects M184V plus additional RT mutations K65R, L74V, and Y115F.

13 Nonclinical Toxicology

13.1 Carcinogenesis, Mutagenesis, Impairment of Fertility

Carcinogenicity

Dolutegravir: Two-year carcinogenicity studies in mice and rats were conducted with dolutegravir. Mice were administered doses of up to 500 mg/kg, and rats were administered doses of up to 50 mg/kg. In mice, no significant increases in the incidence of drug-related neoplasms were observed at the highest doses tested, resulting in dolutegravir AUC exposures approximately 20-fold higher than those in humans at the recommended dose of 50 mg once daily. In rats, no increases in the incidence of drug-related neoplasms were observed at the highest dose tested, resulting in dolutegravir AUC exposures 17 times higher than those in humans at the recommended dose of 50 mg once daily.

Lamivudine: Long-term carcinogenicity studies with lamivudine in mice and rats showed no evidence of carcinogenic potential at exposures up to 12 times (mice) and 72 times (rats) the human exposures at the recommended dose of 300 mg.

Mutagenicity

Dolutegravir: Dolutegravir was not genotoxic in the bacterial reverse mutation assay, in a mouse lymphoma assay, or in the in vivo rodent micronucleus assay.

Lamivudine: Lamivudine was mutagenic in an L5178Y mouse lymphoma assay and clastogenic in a cytogenetic assay using cultured human lymphocytes. Lamivudine was not mutagenic in a microbial mutagenicity assay, in an in vitro cell transformation assay, in a rat micronucleus test, in a rat bone marrow cytogenetic assay, and in an assay for unscheduled DNA synthesis in rat liver.

Impairment of Fertility

Dolutegravir or lamivudine did not affect male or female fertility in rats at doses associated with exposures approximately 33 or 112 times, respectively, higher than the exposures in humans at the doses of 50 mg and 300 mg, respectively.

14 Clinical Studies

14.1 Clinical Trials in Adult Subjects

The efficacy of the combination is supported by data from 2 randomized, double-blind, controlled trials (GEMINI-1 [NCT204861] and GEMINI-2 [NCT205543]) in antiretroviral treatment-naive adults.

GEMINI-1 and GEMINI-2 are identical 148-week, Phase 3, randomized, multicenter, parallel-group, non-inferiority trials. A total of 1,433 HIV-1 infected antiretroviral treatment-naive adult subjects received treatment in the trials. Subjects were enrolled with a screening plasma HIV-1 RNA of 1,000 to ≤500,000 copies/mL, and without evidence of major resistance-associated mutations. Subjects were randomized to receive a 2-drug regimen of TIVICAY plus EPIVIR administered once daily or TIVICAY 50 mg plus fixed-dose TRUVADA administered once daily. The primary efficacy endpoint for each GEMINI trial was the proportion of subjects with plasma HIV-1 RNA <50 copies/mL at Week 48 (Snapshot algorithm for the ITT-E population).

At baseline, in the pooled analysis, the median age of subjects was 33 years, 15% female, 68% white, 9% were CDC Stage 3 (AIDS), the median plasma HIV-1 RNA was 4.4 log₁₀ copies/mL, 20% had HIV-1 RNA >100,000 copies/mL, the median CD4+ cell count was 432 cells/mm³, and 8% had CD4+ cell count ≤200 cells/mm³; these characteristics were similar between trials and treatment arms.

The primary endpoint and other outcomes (including outcomes by key baseline covariates) for the pooled GEMINI-1 and GEMINI-2 trials are shown in Table 11. The virologic outcome results for GEMINI-1 and GEMINI-2 were similar to the pooled GEMINI-1 and GEMINI-2 virologic outcome results.

TABLE 11
Pooled Virologic Outcomes of Randomized Treatment in GEMINI-1 and GEMINI-
2 Trials at Week 48 (Snapshot Algorithm)

| | GEMINI-1 and GEMINI-2 | |
|---|---|---|
| | Pooled Data^{a} | |
| | TIVICAY plus | TIVICAY plus |
| | EPIVIR | TRUVADA |
| Virologic Outcomes | (n = 716) | (n = 717) |
| HIV-1 RNA <50 copies/mL | 91% | 93% |
| Treatment Difference^{b} | −1.7% | |
| | (95% CI: −4.4%, 1.1%) | |
| Virologic nonresponse | 3% | 2% |
| Reasons | | |
| Data in window not <50 copies/mL | 1% | <1% |
| Discontinued for lack of efficacy | <1% | <1% |
| Discontinued for other reasons and ≥50 copies/mL | <1% | <1% |
| Change in ART | <1% | <1% |
| No virologic data at Week 48 window | 6% | 5% |
| Reasons | | |
| Discontinued trial due to adverse event or death | 1% | 2% |
| Discontinued trial for other reasons | 4% | 3% |
| Missing data during window but on trial | <1% | 0% |

Proportion (%) of Subjects with HIV-1 RNA <50 copies/mL by Baseline Category

| | % (n/N) | | % (n/N) | |
|---|---|---|---|---|
| Plasma Viral Load (copies/mL) | | | | |
| ≤100,000 | 91% | (526/576) | 94% | (531/564) |
| >100,000 | 92% | (129/140) | 90% | (138/153) |
| Baseline CD4+ (cells/mm³) | | | | |
| ≤200 | 79% | (50/63) | 93% | (51/55) |
| >200 | 93% | (605/653) | 93% | (618/662) |
| Gender | | | | |
| Male | 92% | (555/603) | 94% | (580/619) |
| Female | 88% | (100/113) | 91% | (89/98) |
| Race | | | | |
| White | 93% | (447/480) | 95% | (471/497) |
| African-American/African Heritage/Other | 88% | (208/236) | 90% | (198/220) |
| Age (years) | | | | |
| <50 | 92% | (597/651) | 94% | (597/637) |
| ≥50 | 89% | (58/65) | 90% | (72/80) |

^{a}The results of the pooled analysis are in line with those of the individual trials, for which the primary endpoint (difference in proportion <50 copies/mL plasma HIV-1 RNA at Week 48 based on the Snapshot algorithm for TIVICAY plus EPIVIR versus TIVICAY plus TRUVADA) was met. The adjusted difference was −2.6 (95% CI: −6.7; 1.5) for GEMINI-1 and −0.7 (95% CI: −4.3; 2.9) for GEMINI-2 with a prespecified non-inferiority margin of 10%.
^{b}Based on CMH-stratified analysis adjusting for the following baseline stratification factors: plasma HIV-1 RNA (≤100,000 copies/mL vs. >100,000 copies/mL) and CD4+ cell count (≤200 cells/mm³ versus >200 cells/mm³). Pooled analysis also stratified by trial. Assessed using a non-inferiority margin of 10%.

The adjusted mean change from baseline in CD4+ cell count based on the pooled analysis at Week 48 was 224 cells/mm³ for the group receiving TIVICAY plus EPIVIR, and 217 cells/mm³ for the group receiving TIVICAY plus TRUVADA.

16 How Supplied/Storage and Handling

Each the combination tablet contains 50 mg of dolutegravir as dolutegravir sodium and 300 mg lamivudine and is an oval, biconvex, white, film-coated tablet, debossed with “SV 137” on one face.

Bottle of 30 tablets with child-resistant closure NDC 49702-246-13.

Store up to 30° C. (86° F.).

1. A method of treating HIV in a human in need thereof which comprises the in vivo administration of a therapeutically effective amount of a combination of:
dolutegravir or a pharmaceutically acceptable salt thereof, and
lamivudine or a pharmaceutically acceptable salt thereof

2. The method of claim 1, which comprises the in vivo administration of a therapeutically effective amount of a combination of:
dolutegravir sodium, and
lamivudine or a pharmaceutically acceptable salt thereof.

3. The method of claim 1, wherein the method takes into account adverse reactions of the combination.

4. The method of claim 1, wherein the method takes into account Drug Interactions of the combination.

5. The method of claim 1, wherein the method takes into account the clinical pharmacology of the combination.

6. The method of claim 1, wherein the method takes into account the toxicology of the combination.

7. The method of claim 1, wherein the combination is co-administered with carbamazepine or rifampin and an additional 50mg of dolutegravir or a pharmaceutically acceptable salt thereof is administered separated by 12 hours.

8. The method of claim 1, wherein the combination is co-administered with metformin and the total daily dose of metformin is limited to 1000 mg either when starting metformin or the combination.

9. The method of claim 1, wherein the combination is administered 2 hours before or 6 hours after taking medications containing polyvalent cations.

10. The method of claim 9, wherein the medication containing polyvalent cations contains magnesium or aluminum.

11. The method of claim 9, wherein the medication containing polyvalent cations is a cation-containing antacid or laxative, sucralfate or a buffered medication.

12. The method of claim 1, wherein the medication is administered 2 hours before or 6 hours after taking supplements containing calcium or iron.

13. The method of claim 1, wherein the medication is co-administered with supplements containing calcium or iron together with food.
